(19) United States
(12) Patent Application Publication
(10) Pub. No.: US 2005/0059922 A1
(43) Pub. Date: Mar. 17, 2005

(54) Surgical implant and method of accessing cerebrospinal fluid

(76) Inventors: Steven Michael Kuhlman, Richland, MI (US); Matthew Crosby Flegal, Kalamazoo, MI (US); Donna Jean Waggoner, Kalamazoo, MI (US); Allen Edwin Buhl, Kalamazoo, MI (US)

Correspondence Address: FLYNN, THIEL, BOUTELL & TANIS, P.C., 2026 RAMBLING ROAD, KALAMAZOO, MI, 49008-1699, US

(21) Appl. No.: 10/900,258

(22) Filed: Jul. 27, 2004

Related U.S. Application Data
(60) Provisional application No. 60/492,176, filed on Aug. 1, 2003.

(51) Int. Cl. A61M 5/00

(52) U.S. Cl. 604/8

(57) ABSTRACT

A surgical implant and method of gaining access to cerebrospinal fluid in the brain. The implant includes a housing defined by an upper housing part which is positioned subcutaneously on the skull and a lower housing part which projects through an opening defined in the skull. The housing defines therein a reservoir which communicates with cerebrospinal fluid located under the skull. The cerebrospinal fluid is accessed for sampling or dosing purposes through a septum mounted on the housing.

### FIELD OF THE INVENTION

This invention generally relates to an implant and method of gaining access to cerebrospinal fluid (csf) in the brain.

### BACKGROUND OF THE INVENTION

It is often necessary for research and treatment purposes to have access to cerebrospinal fluid from conscious patients. For example, by sampling or collecting cerebrospinal fluid, the progression of various brain diseases, infections, or other ailments can be monitored on a regular basis. In research, cerebrospinal fluid sampling is often required to monitor drug levels as well as to monitor changes in physiological parameters in the cerebrospinal fluid. Further, it is often desirable or necessary to administer therapeutic agents directly into the cerebrospinal fluid to bypass the blood-brain barrier.

Various devices and methods have been developed for the purpose of accessing cerebrospinal fluid in animals. One such device is a guide cannula which is implanted within the skull of the animal. One or more of these guide cannulas are secured into the skull of the animal and extend to touch the surface of the dura mater on the surface of the brain so that each of the guides is aligned (but not in contact) with one of the lateral ventricles of the brain. The guides are implanted for the purpose of permitting repeated sampling of cerebrospinal fluid over a predetermined span of time, and thus the guides are left within the skull of the animal and are accessed via a collection needle placed through the skin and muscle located above the respective guides following a surgical-style preparation of the skin over the guides. The needle is inserted into the guide cannula and is guided thereby into the corresponding lateral ventricle to collect cerebrospinal fluid. One of the disadvantages of this arrangement is that the guide cannula locks to the skull of the animal with screw-threads, which can cause difficulty with respect to successfully aligning the needle guide in relation to the lateral ventricle. Further, the screw-threads often result in improper placement of the guide cannula when the sloped surface of the skull catches the threads and pulls the implant out of proper alignment. Another disadvantage of the above arrangement is that same is typically not suitable for use on small animals due to the extremely small size of the lateral ventricles and thinness of the skull.

The present invention is directed to an implant for accessing cerebrospinal fluid from the brain. The implant includes a housing defined by an upper housing part which is fixed to the skull, and a lower housing part which protrudes from the upper housing part through a hole in the skull and dura mater. In a preferred embodiment, the implant is positioned over a space, called the transverse fissure, defined in the brain between the cerebrum and the cerebellum where pools of cerebrospinal fluid are located. The housing defines a reservoir for cerebrospinal fluid, and since cerebrospinal fluid is under pressure within the brain, this fluid flows into the lower housing part and up into the reservoir. The upper part of the housing is closed off with a septum, and cerebrospinal fluid is accessed and withdrawn with a collection needle which is used to penetrate the septum through the skin to collect fluid from the reservoir. Alternatively, therapeutic agents can be dosed directly into the cerebrospinal fluid with a dosing needle which penetrates the septum and delivers the drug into the reservoir for circulation. The implant according to the invention thus serves to create a closed system over a surgically-created opening in the skull and dura mater so as to create a reservoir or access port for cerebrospinal fluid from around the brain.

Other objects and purposes of the invention will be apparent to persons familiar with devices of this type upon reading the following description and inspecting the accompanying drawings.

### BRIEF DESCRIPTION OF THE DRAWINGS

FIG. 1 is front elevational view of the implant according to the invention.

FIG. 2 is a plan view of the implant.

FIG. 3 is a cross-sectional view of the implant taken generally along line 3-3 in FIG. 2.

FIG. 4 is a plan view of the brain of an animal.

FIG. 5 is an elevational side view of the brain of FIG. 4.

FIG. 6 is an enlarged, cross-sectional view of the implant in position in the brain of the animal.

FIG. 7 is a front elevational view of a second embodiment of the implant according to the invention.

FIG. 8 is a plan view of the second embodiment.

FIG. 9 is a cross-sectional view of the implant of the second embodiment taken generally along lines 9-9 in FIG. 8.

FIG. 10 is an enlarged, cross-sectional view of the second embodiment in position in the brain of the animal.

Certain terminology will be used in the following description for convenience in reference only, and will not be limiting. For example, the words “upwardly”, “downwardly”, “rightwardly” and “leftwardly” will refer to directions in the drawings to which reference is made. The words “front” and “rear” will be used to refer to the spatial orientation of components of the implant in relation to the anatomical front and rear of the animal, respectively. The words “inwardly” and “outwardly” will refer to directions toward and away from, respectively, the geometric center of the arrangement and designated parts thereof. Said terminology will include the words specifically mentioned, derivatives thereof, and words of similar import.

### DETAILED DESCRIPTION

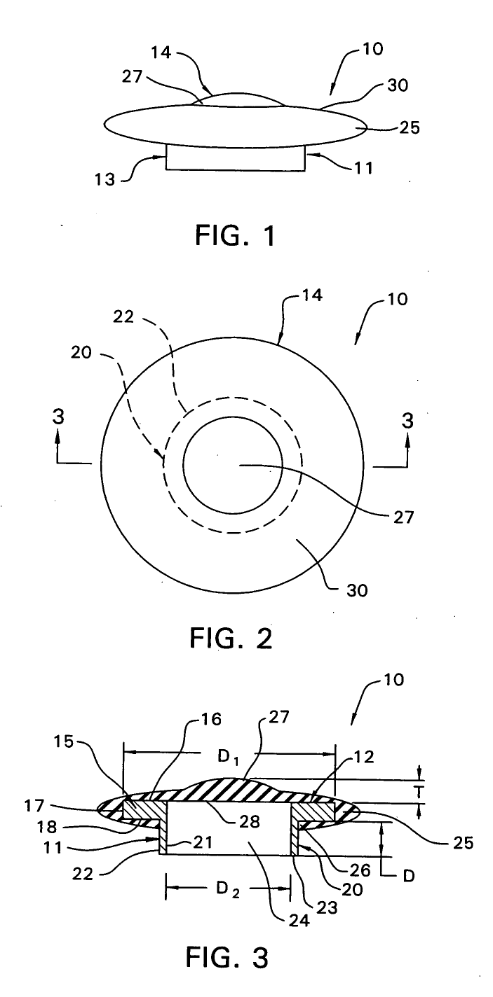

Referring to FIGS. 1-3, an implant 10 is illustrated according to the present invention. The implant 10 generally includes a rigid housing 11 defined by an upper housing part 12 and a lower housing part 13 which depends downwardly from upper housing part 12, and a septum 14 secured to upper housing part 12.

Upper housing part 12 is defined by a generally flat, plate-like and annular wall 15. Wall 15 defines a generally planar upper surface 16, an outer annular side surface or edge 17 which is generally perpendicular to surface 16, and a lower surface 18 which is generally parallel to upper surface 16. Lower housing part 13 is defined by a cylinder 20 having an outer diameter which is less than an outer diameter of wall 15, and which cylinder 20 defines respective inner and outer generally vertical and parallel surfaces 21 and 22. Cylinder 20 projects downwardly from lower surface 18 of wall 15 and terminates in a lower generally horizontal edge 23 which extends transversely between surfaces 21 and 22. The wall 15 and cylinder 20 in the illustrated embodiment are integrally formed with one another, and together define a reservoir in the form of a bore 24. Reservoir 24 in the illustrated embodiment has a substantially constant diameter throughout the length or vertical extent thereof.

Septum 14 is mounted on upper housing part 12, and includes an annular side wall 25 which wraps essentially completely around and is disposed in surrounding relation with wall 15 of housing part 12. More specifically, side wall 25 extends over surfaces 16, 17 and 18 of wall 15, and has a terminal edge portion 26 which is disposed closely adjacent, and in the illustrated embodiment, in contacting relation with outer surface 22 of cylinder 20. Septum 14 additionally includes a rounded upper portion or head 27 which is integrally formed with side wall 25 and which projects slightly upwardly from an upper surface 30 of side wall 25. A lower and generally planar surface 28 of head 27 defines the uppermost extent of reservoir 24, as shown in FIG. 3.

In the illustrated embodiment, the housing 11, including upper housing part 12 and lower housing part 13, is constructed of surgical-grade stainless steel. However, housing 11 may alternatively be constructed of non-reactive, injection-molded plastic, resin or titanium. The septum 14 is constructed of silicone or other non-reactive rubber materials, and in the illustrated embodiment can be secured to housing 11 through a conventional molding process and/or adhesive. Septum 14 may also be secured to housing 11 through a mechanical interlock arrangement.

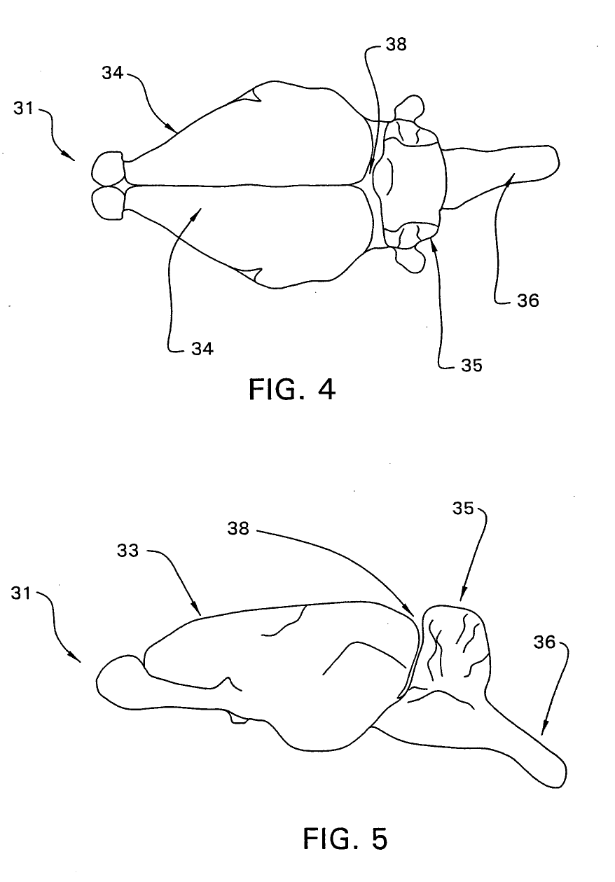

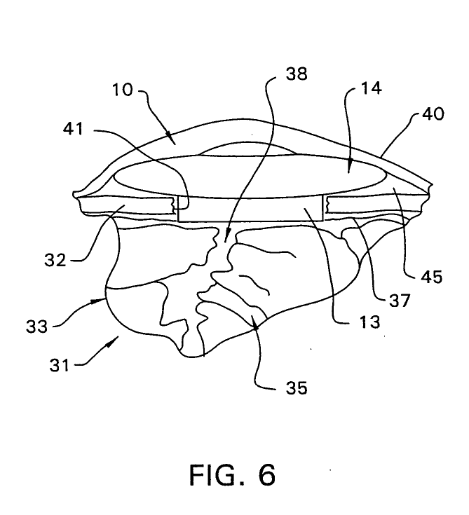

FIGS. 4-6 illustrate a brain 31 of a guinea pig. The brain 31 is contained within the skull 32 (shown only partially in FIG. 6), and includes three primary parts, the cerebrum 33 which is defined by the cerebral hemispheres 34, the cerebellum 35 and the spinal cord or brain stem 36. A thick and fibrous membrane called the dura mater 37 (shown in FIG. 6 only) lines the interior of the skull 32. As best shown in FIG. 6, an area of the brain 31 called the transverse fissure 38 is defined between the cerebrum 33 and the cerebellum 35.

The most common sites for accessing cerebrospinal fluid in the brain are the lateral ventricles (not shown). However, cerebrospinal fluid bathes the entire surface of the brain and tends to pool or collect at various sites within the brain and closer to the skull 32 beneath the dura mater 37, and at least one of these sites is located adjacent the transverse fissure 38 mentioned above. The implant 10 according to the invention thus utilizes the transverse fissure 38 to access cerebrospinal fluid as discussed below.

The device 10 according to the invention is implanted within the brain 31 of the animal as follows, with reference to FIG. 6. If desirable or necessary, the animal can be placed in a stereotaxic device in order to identify the precise location on the skull 32 for transection by the surgeon. The skin 40 is incised along the top of the head, and is retracted and musculature is stripped away from the midline of the skull 32. A pilot hole is drilled through the skull until the dura mater 37 is reached, and an operating microscope is utilized at this juncture and the hole is adjusted as necessary so that the space between the cerebellum 35 and cerebrum 33 can be visualized. The hole is then extended through the skull 32 so as to define an opening 41 which is large enough to allow passage of the lower housing part 13 into the skull 32, and then the dura mater 37 is incised and removed to allow insertion of the housing part 13 with the position being modified as needed to avoid major blood vessels. A thin bead of surgical-grade cyanoacrylate gel is placed around the rim of the opening 41 and the implant 10 is pushed into place into the gel above the transverse fissure 38 until the wall 15 and the surrounding side wall 25 of the septum 14 rest against the outer surface of the skull 32. An adhesive 45, such as dental acrylic, is utilized to build up the skull 32 around the septum 14 to lock the implant 10 into place on the skull 32. The skin 40 is then sutured so that same completely covers the septum 14.

Since cerebrospinal fluid is under pressure in the brain 31, this fluid will flow into the lower open end of the cylinder 20 and into the reservoir 24. Cerebrospinal fluid has it's own currents which serve to keep the fluid continuously flowing in and out of the reservoir 24, thereby preventing stagnation of the fluid. When sampling of cerebrospinal fluid is desirable or necessary, the skull 32 of the animal is felt with the fingers in order to locate the bump or nodule created by the upper housing part 12 and septum 14 of the implant 10. Using standard aseptic practices (all personnel wearing surgical masks and bonnets, the use of sterile surgical-style skin preparation involving 3-5 alternating scrubs with povidone iodine or chlorhexidene soaps followed by 70% isopropyl alcohol and a final application of povidone iodine solution or film), a collection needle is then pushed through the skin 40, through the septum 14 and into the reservoir 24, and cerebrospinal fluid is withdrawn therefrom into the needle. New cerebrospinal fluid will then replace that which was removed from the reservoir 24. The same procedure is utilized when dosing of a drug or drugs is desirable or necessary, except that a dosing needle is utilized instead of a collection needle and serves to deliver a drug or drugs directly into the cerebrospinal fluid via the reservoir 24.

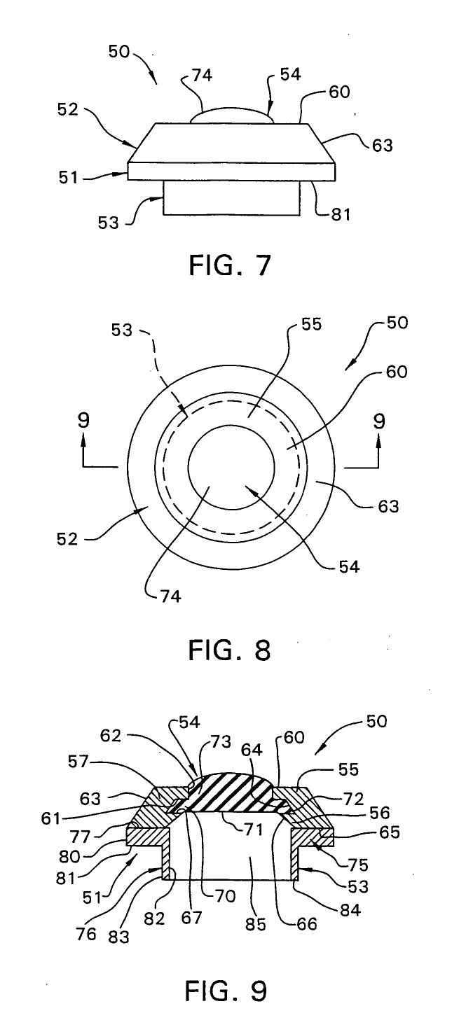

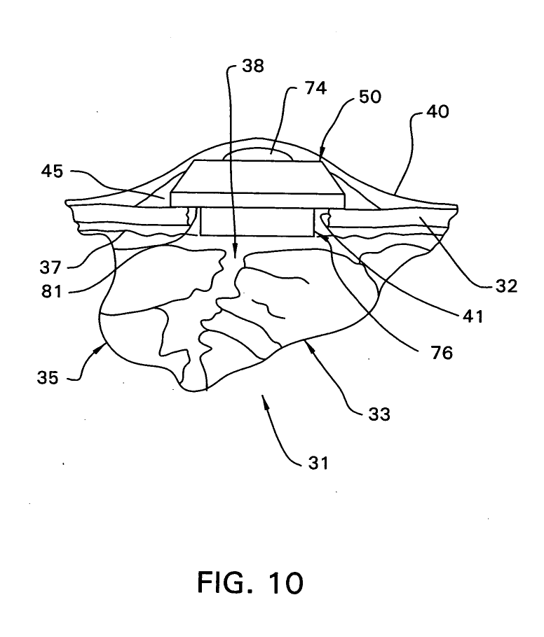

Another embodiment of the invention is illustrated in FIGS. 7-10, which may also be used with a small animal, such as a guinea pig. The implant 50 pursuant to the second embodiment generally includes a rigid housing 51 defined by an upper housing part 52 and a lower housing part 53, and a septum 54 mounted on upper housing part 52.

Upper housing part 52 defines a generally flat and annular upper wall 55 which is vertically spaced from and generally parallel with a generally flat and annular lower wall 56. An annular side wall 57 extends between and adjoins upper and lower walls 55 and 56. Side wall 57 angles outwardly as same projects downwardly from upper wall 55 towards lower wall 56, and has a frusto-conical shape when viewed from the side.

Upper wall 55 defines a flat and annular upper surface 60, a flat and annular lower surface 61 which is vertically spaced from upper surface 60, and an inner terminal edge surface 62 which is generally vertically oriented and extends between upper and lower surfaces 60 and 61. Side wall 57 defines a tapered outer surface 63 which adjoins upper surface 60, and a tapered inner surface 64 which is generally parallel to outer surface 63. Lower wall 56 defines a flat and annular lower surface 65 which adjoins outer surface 63, an inner inclined surface 66 which is spaced inwardly from, but generally parallel to inner surface 64 of side wall 57, and an upper surface 67 which is generally parallel to lower surface 65. Upper housing part 52 thus defines therein a stepped recess which generally has the shape of an inverted “T” when viewed in cross-section and is defined by surfaces 61, 62, 64 and 67, with the narrowest or smallest cylindrical part of this recess being disposed uppermost and defined by surface 62.

Septum 14 is mounted or embedded within the recess of upper housing part 52. Septum 14 includes a lower flange 70 having a generally flat bottom surface 71, an annular side surface 72 which projects upwardly from bottom surface 71 and is generally parallel to surface 63 of side wall 57. A cylindrical part 73 of septum 14 projects upwardly from lower flange 70 and terminates in a rounded head 74. The septum 14 in the illustrated embodiment is mechanically interlocked with upper housing part 52 through the engagement of lower flange 70 within the lower sidewardly projecting portion of the housing recess defined by surfaces 67, 64 and 61, with the cylindrical part 73 of septum 14 being disposed within the upper cylindrical part of the recess. When mounted on upper housing part 52, the rounded head 74 of septum 14 projects vertically upwardly slightly beyond the upper surface 60 of upper wall 55. As mentioned above, the septum 14 in the illustrated embodiment is mechanically interlocked with housing part 52 through the engagement of lower flange 70 within the housing recess, and if desirable or necessary, septum 14 can be further secured to housing part 52 with adhesive.

Lower housing part 53 includes a generally flat, plate-like and annular wall 75, and a cylinder 76 which depends downwardly therefrom. Wall 75 defines a generally planar upper surface 77, an outer annular side surface or edge 80 which is generally perpendicular to surface 77, and a lower surface 81 which is generally parallel to upper surface 77. Cylinder 76 has an outer diameter which is less than an outer diameter of wall 75, and which cylinder 76 defines respective inner and outer generally vertical and parallel surfaces 82 and 83. Cylinder 76 projects downwardly from lower surface 81 of wall 75 and terminates in a lower generally horizontal and annular edge 84 which extends transversely between surfaces 82 and 83. The wall 75 and cylinder 76 in the illustrated embodiment are integrally formed with one another, and along with inclined surface 66 of lower wall 56 of upper housing part 52 define a reservoir in the form of a bore 85. Reservoir 85 in the illustrated embodiment has a substantially constant diameter throughout the length or vertical height thereof, except that same has a somewhat narrowed upper diameter defined by inclined surface 66. As shown in FIG. 9, the bottom surface 71 of septum 14 defines the uppermost extent of reservoir 85.

The upper and lower housing parts 52 and 53 are secured to one another with suitable epoxy or adhesive which is placed between lower surface 65 of lower wall 56 and upper surface 77 of wall 75.

The implant 50 according to the second embodiment of the invention is implanted within the brain 31 of the animal above the transverse fissure 38 in a similar manner as discussed above with regard to the first embodiment, with the lower surface 81 of lower housing part 53 resting against the upper surface of the skull 32, and the cylinder 76 projecting into the opening 41 formed in the skull 32 and dura mater 37. Cerebrospinal fluid can be sampled or a drug or drugs can be administered through the septum 54 as discussed above.

In the second embodiment, the housing 51, including upper housing part 52 and lower housing part 53, is constructed of surgical-grade stainless steel, but may alternatively be constructed of non-reactive, injection-molded plastic, resin or titanium. The septum 54 is constructed of silicone or other non-reactive rubber materials.

The implants 10 and 50 according to the invention are ideal for use on small animals, particularly since the lateral ventricles and cisterna magna which are often utilized to sample or dose cerebrospinal fluid are quire small, and are thus extremely difficult to access via conventional devices. The implants disclosed herein instead create a port through which cerebrospinal fluid, which tends to pool in areas under the dura mater of the brain, can be repeatedly accessed in a conscious animal without the need for anesthesia, and create less trauma and discomfort to the animal than conventional methods.

The implants 10 and 50 according to the invention are described herein for use with small animals such as guinea pigs, rats and rabbits. However, it will be appreciated that the device can be utilized with other larger animals, such as canines. In this regard, the implants illustrated herein can be scaled up for use on a larger animal such as a canine, and the size of the implant would be approximately four times larger than the implant for use with a guinea pig or other similarly sized animal. It is also contemplated that the implants according to the invention can be used in humans, and thus can be scaled accordingly.

As discussed above, the implant according to the invention can be utilized with different animals, and also humans, and thus the size thereof will be based upon the brain size and structure of the particular animal. With respect to the first embodiment for use with a small animal such as a guinea pig and with reference to FIG. 3, the outer diameter D1 of the upper housing part 12 is approximately 8 mm, the diameter D2 of the reservoir 24 is approximately 5 mm, the height H of the lower housing part 13 is approximately 1-2 mm, and the thickness T of the septum 14 is approximately 1 mm. It will be appreciated that the above dimensions are given by way of example only, and are not to be limiting. The dimensions of the second embodiment of the implant 50 would be similar to those given above.

Although particular preferred embodiments of the invention are disclosed in detail for illustrative purposes, it will be recognized that variations or modifications of the disclosed apparatus, including the rearrangement of parts, lie within the scope of the present invention.

1. An implant for accessing cerebrospinal fluid in the brain which includes a cerebrum and a cerebellum enclosed by a skull, said implant comprising a housing including an upper housing part for positioning on the skull, and a lower housing part depending downwardly from said upper housing part for projection through an opening defined in the skull, a septum mounted on said housing, said housing defining therein a reservoir adjacent said septum for communication with cerebrospinal fluid located under the skull which is accessible through said septum.

2. The implant of claim 1 wherein said lower housing part has an outer diameter which is less than an outer diameter of said upper housing part.

3. The implant of claim 1 wherein said reservoir is defined within said upper and lower housing parts.

4. The implant of claim 1 wherein said septum is mounted on said upper housing part.

5. The implant of claim 4 wherein said upper housing part includes a flat, plate-like annular member and said lower housing part includes a cylinder which projects downwardly from said annular member, said annular member and said cylinder defining respective inner annular surfaces which together define said reservoir, said reservoir extending completely through said housing.

6. The implant of claim 5 wherein said septum is disposed in surrounding relation with said annular member and extends over an upper portion thereof to close off and define an upper end of said reservoir.

7. The implant of claim 4 wherein said septum is embedded within and interlocked with said upper housing part.

8. The implant of claim 7 wherein said upper housing part comprises an annular member and said lower housing part includes an annular flange engaged with a lower surface of said annular member and a cylinder which projects downwardly from said flange, said upper and lower housing parts defining respective inner annular surfaces which together define said reservoir which extends completely through said housing, and said septum is disposed to close off and define an upper end of said reservoir.

9. The implant of claim 1 wherein said lower housing part is threadless and is secured within the opening in the skull with adhesive.

10. The implant of claim 1 wherein said reservoir opens downwardly through said lower housing part and directly communicates with a pool of cerebrospinal fluid located under the skull such that the cerebrospinal fluid travels directly from the pool and upwardly into said reservoir.

11. A surgical implant positioned subcutaneously on the skull of a small animal for providing access to cerebrospinal fluid in the brain of the animal, said implant comprising a septum and defining a reservoir adjacent said septum, a rigid upper portion having a lower surface positioned on the skull, and a rigid lower portion connected to said upper portion and projecting outwardly therefrom, said lower portion being positioned within an opening defined in the skull and said reservoir opening through said lower portion for communication with a pool of cerebrospinal fluid located under the skull which is accessible through said septum.

12. The implant of claim 11 wherein said lower portion has a reduced outer dimension as compared to an outer dimension of said upper portion.

13. The implant of claim 12 wherein said upper and lower portions are generally circular in shape and said outer dimensions thereof are diameters.

14. The implant of claim 11 wherein said reservoir extends completely through said upper and lower portions and is closed off at an upper end thereof by said septum which is mounted on said upper portion and is penetrable to permit dosing of therapeutic agents into the cerebrospinal fluid or to permit collection of cerebrospinal fluid from said reservoir.

15. The implant of claim 11 wherein said septum is mounted on said upper portion.

16. A method of surgically inserting an implant into the brain of a patient, said method comprising the steps of:
providing an implant having a housing defining a reservoir therein, an upper housing part, a lower housing part cantilevered downwardly from the upper housing part, and a septum adjacent the reservoir;
locating an area of the brain over the transverse fissure defined between the cerebrum and the cerebellum which contains cerebrospinal fluid;
forming an opening in the skull at said area;
inserting the lower housing part into the opening until the upper housing part is positioned on the skull; and
securing the housing to the skull with adhesive, and without the use of threads on the housing.

17. The method of claim 16 including surgically inserting the implant in the brain of a small animal, such as a guinea pig.

18. A method of accessing cerebrospinal fluid from the brain, said method comprising the steps of:
providing an implant having a housing defining a reservoir therein, an upper housing part, a lower housing part cantilevered downwardly from the upper housing part, and a septum adjacent the reservoir;
embedding the implant within the brain so that the lower housing part projects through an opening defined in the skull adjacent a pool of cerebrospinal fluid, and so that the upper housing part is positioned subcutaneously on the skull; and
accessing cerebrospinal fluid within the reservoir through the septum.

19. The method of claim 18 including inserting one of: a collection needle through the septum and into the reservoir and collecting cerebrospinal fluid; and a dosing needle through the septum and into the reservoir and dosing the cerebrospinal fluid with a therapeutic agent.

20. The method of claim 18 wherein said step of embedding includes embedding the implant within the brain so that the lower housing part projects through an opening defined in the skull adjacent the transverse fissure defined between the cerebrum and the cerebellum where cerebrospinal fluid pools.
